SEQUENCE LISTING (1 sequence)

SEQ ID NO: 1 - protein, 22 aa, Artificial Sequence
  Synthetic Construct
ZRCCNGRRGCSSRWCRDHSRCC

(19) United States
(12) Patent Application Publication
(10) Pub. No.: US 2011/0304329 A1
(43) Pub. Date: Dec. 15, 2011

(54) LABELED PEPTIDES AND METHODS OF USE THEREOF FOR IMPROVED OXIDATION AND MAPPING OF DISULFIDE BRIDGES

(75) Inventors: Grzegorz Bulaj, Salt LakeCity, UT (US); Aleksandra Walewska, Salt Lake City, UT (US); Jack J. Skalicky, Salt Lake City, UT (US); Baldomero M. Olivera, Salt Lake City, UT (US); Eric W. Schmidt, Salt Lake City, UT (US); Darrell R. Davis, Salt Lake City, UT (US)

(73) Assignee: University of Utah Foundation, Salt Lake City, UT (US)

(21) Appl. No.: 13/060,329

(22) PCT Filed: Aug. 26, 2009
(86) PCT No.: PCT/US09/55020

§ 371 Date: Sep. 2, 2011

Related U.S. Application Data
(60) Provisional application No. 61/092,426, filed on Aug. 28, 2008.

(51) Int. Cl. G01R 33/44; C07K 14/00

(52) U.S. Cl. 324/309; 530/326; 530/323; 530/322

(57) ABSTRACT

Described herein are labeled proteins and methods of use thereof for identifying the position of multiple disulfide bridges present in the peptide. The methods combine the use of diselenide bridges and NMR-based mapping of the disulfide bridges. Also described herein are labeled proteins described above that contain fluorous bridges and spacers that facilitate oxidative folding of the protein. The resulting biorthogonal oxidation strategy for studying disulfide-rich peptides both improves oxidative folding and provides simultaneous determination of the disulfide crosslink connectivity in the peptide. The methods permit routine and facile production of disulfide-rich peptides.

### CROSS REFERENCE TO RELATED APPLICATIONS

This application claims priority upon U.S. provisional application Ser. No. 61/092,426, filed Aug. 28, 2008. This application is hereby incorporated by reference in its entirety for all of its teachings.

### ACKNOWLEDGEMENTS

The research leading to this invention was funded in part by the National Institutes of Health, Grant Nos. R21NS055845 and Program Project GM 48677. The U.S. Government has certain rights in this invention.

### BACKGROUND

Short cysteine-rich peptides, such as neurotoxins from venomous spiders, scorpions, cone snails, plant-derived cyclotides or proteinase inhibitors are a megadiverse group of natural products, composed of many estimated millions of distinct sequences, only a miniscule fraction of which have been characterized. For a comprehensive characterization (including structure/function studies) of cysteine-rich peptides, efficient synthetic or recombinant methods are required. In particular, a strategy for evaluating oxidative folding is important. Achieving the correct disulfide connectivity is a major barrier that needs to be overcome in the chemical synthesis of any disulfide-rich peptide.

Numerous strategies have been developed to improve the oxidative folding of cysteine-rich polypeptides. The immobilized Ellman's reagent (currently available as a Clear-Ox) can be used to oxidize one-, two-, and three-disulfide bridged peptides. Once the folded disulfide-rich peptide has been synthesized, evidence is needed that the correct connectivity of disulfide bridges has been achieved. Currently, testing bioactivity and mass spectrometry-based peptide mapping are used to infer that the synthetic peptide has the native connectivity. Therefore, there is a need for a methodology that can unambiguously assign multiple disulfide linkages in a peptide produced by oxidative folding. The methodology would provide a powerful tool in the screening and design of new drugs.

### SUMMARY

Described herein are labeled proteins and methods of use thereof for identifying the position of multiple disulfide bridges present in the peptide. The methods combine the use of diselenide bridges and NMR-based mapping of the disulfide bridges. Also described herein are labeled proteins described above that contain fluorous bridges and spacers that facilitate oxidative folding of the protein. The resulting biorthogonal oxidation strategy for studying disulfide-rich peptides both improves oxidative folding and provides simultaneous determination of the disulfide crosslink connectivity in the peptide. The methods permit routine and facile production of disulfide-rich peptides. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows (A) an example of a peptide having two disulfide bridges and one diselenide bridge and evaluation of the position of the disulfide bridges and (B) the design of μ-selenoconotoxin analogs of SIIIA.

FIG. 2 shows examples of peptides containing more than three disulfide bridges formed by oxidative folding of cysteine-rich peptides.

FIG. 3 shows the HPLC chromatograms (A and B) and yields of μ-selenoconotoxin SIIIA analogs (C and D).

FIG. 4 shows the blocking of Nav1.2 sodium channels by μ-selenoconotoxin SIIIA analogs.

FIG. 5 shows the structures, folding and the properties of “nonnatural” analogs of μ-selenocontoxin SIIIA: (A) structure of AHX-Sec-SIIIA; (B) HPLC separation of the folding reaction, star indicated the peak that was further characterized in the electrophysiology assay; (C) activity of AHX-Sec-SIIIA in blocking Nav1.2 currents; (D) structure of DOTA-Sec-SIIIA analog; (E) HPLC separation of the folding reaction of DOTA-Sec-SIIIA, star indicated the peak that was further characterized in the electrophysiology assay and loaded with terbium; and (F) excitation and emission spectra.

FIG. 6 shows (A) the folding of μ-selenoconotoxin SmIIIA analog and (B) the ability of μ-selenoconotoxin SmIIIA analog to block of Nav 1.2 sodium channels.

FIG. 7 shows the NMR-based determination of the disulfide bridging pattern in two μ-selenoconotoxin SIIIA analogs: SIIIA[C3U,C13U,*C4,19] and SIIIA[*C3,13,C4U,C19U].

FIG. 8 shows Sec-GVIA analogs and strategy for disulfide connectivity determination. (a) The position of distinct diselenide bond in 3D-structure of GVIA (diselenide bond in grey and disulfide bond in yellow) and corresponding scaffold (U and C corresponds to selenocysteine and labeled cysteine, respectively). Structure of GVIA (2CCO) was processes using PyMOL software. (b) Demonstrates the concept of integrated oxidative folding using GVIA [C8U C19U]. Redox potential favors diselenide (−381 mV) over selinysulfide (−326 mV) and labeled cysteine residues help in rapid identification of cross-disulfide NOEs.

FIG. 9 shows the synthesis and oxidative folding of GVIA and Sec-GVIA analogs. (a) RP-HPLC elution profiles of linear and folded peptides. Linear Sec-GVIA analogs contain a diselenide, formed during the processing of peptide. Folded peptide profile corresponds to the oxidative folding at steady-state. Asterisk indicates the natively folded peptide. (b) Accumulation of natively folded peptide during oxidative folding at steady-state. Error bars represents standard error of mean, derived from four independent experiments.

FIG. 10 shows the evaluation of the quality of folded peptide having identical retention time with that of reduced linear disulfide rich peptide using high resolution mass spectrometry. FT-mass spectra of GIVA[C1U C16U]: (a) Reduced linear peptide; (b) mixture of equal amount of reduced/folded peptide; (c) folded peptide and (d) theoretical mass spectrum of folded peptide derived from elemental composition. Shown mass spectra corresponds to +4 charge state ([M+4H]⁴⁺). Mass spectrum for the folded peptide GVIA[C1U C16U], derived at 1 min time interval during folding kinetics, identical to that of folded peptide in isotopic pattern confirming the absence of residual reduced peptide.

FIG. 11 shows NMR based disulfide mapping in Sec-GVIA analogs. Alignment of 2D F2-¹³C-edited [¹H,¹H] NOESY (panels a, c, & e) with corresponding 2D [¹³C,¹H] HSQC (panels b, d, & f) spectra of Sec-GVIA analogs: GVIA[C1U C16U](panels a & b), GVIA[C8U C19U](panel c & d) and GVIA[C15U C26U](panel e & f). Non-degenerate Cys C^{β}H₂ are connected with a line and NOE cross peaks confirming the disulfide bond between labeled cysteines are boxed. NOE between C1^{α}H/C16^{α}H in GVIA[C15U C26U] is detected on the sharp C1^{α}H signal only and not on the broader C16^{α}H signal. The NOEs not labeled are assigned as intra-cysteine or from unassigned proximal protons, which pose no problems in disulfide mapping.

FIG. 12 shows the blocking effectiveness of GVIA and Sec-GVIA analogs. N-type currents recorded were before (grey) and after (black) 16 min application of the conopeptides, which were GVIA (a), GVIA[C1U C16U] (b), GVIA[C8U C19U] (c), and GVIA[C15U C26U]. Voltage protocol is shown at the bottom of each panel. The tail currents on the control records were clipped to highlight the block of step current.

FIG. 13 shows the circular dichroism spectra of GVIA and Sec-GVIA analogs. (a) Folded peptides, Sec analogues contain a diselenide and two disulfides whereas GVIA contains three disulfides. Spectra were recorded at pH 6.8. (b) Linear peptides, Sec analogues contain a diselenide and four free thiols, whereas GVIA contains six free thiols. Spectra were recorded in 1 mM DTT at pH 8.0. Shown is an average spectra obtained from five independent scans.

FIG. 14 shows the folding kinetics of GVIA and Sec-GVIA analogues. (a) Reverse-phase C₁₈ analytical HPLC elution profiles of the oxidative folding pathway. Reactions were quenched by acidification at regular intervals of time and analyzed using chromatography. The major peak in the profile indicates the natively folded peptide. (b) The accumulation of folded peptides was plotted against the regular intervals of time. Rate constants were obtained by fitting the experimental points to single exponential fit. (c) Bar graph summarizing the time required to fold fifty percent of the natively folded peptide.

FIG. 15 shows a schematic representation of the oxidative folding summary of GVIA and Sec-GVIA analogs. The topology of Sec-GVIA analogs were presented and number of residues involved in the loop was also indicated. Folding yield derived from steady-state experiment and kinetics of oxidative folding to native form were emphasized.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optional spacer” means that the spacer may or may not be present in the peptide.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The term “polyalkylene group” as used herein is a group having two or more CH₂ groups linked to one another. The polyalkylene group can be represented by the formula —(CH₂)ₙ—, where n is an integer of from 2 to 25.

The term “polyether group” as used herein is a group having the formula —[(CHR)ₙO]ₘ—, where R is hydrogen or a lower alkyl group, n is an integer of from 1 to 20, and m is an integer of from 1 to 100. Examples of polyether groups include, polyethylene oxide, polypropylene oxide, and polybutylene oxide.

The term “polythioether group” as used herein is a group having the formula —[(CHR)ₙS]ₘ—, where R is hydrogen or a lower alkyl group, n is an integer of from 1 to 20, and m is an integer of from 1 to 100.

The term “polyimino group” as used herein is a group having the formula —[(CHR)ₙNR]ₘ—, where each R is, independently, hydrogen or a lower alkyl group, n is an integer of from 1 to 20, and m is an integer of from 1 to 100.

The term “polyester group” as used herein is a group that is produced by the reaction between a compound having at least two carboxylic acid groups with a compound having at least two hydroxyl groups.

The term “polyamide group” as used herein is a group that is produced by the reaction between a compound having at least two carboxylic acid groups with a compound having at least two unsubstituted or monosubstituted amino groups.

The term “peptide” is any compound produced from a plurality of amino acids. Examples of peptides include natural and synthetic proteins, peptoids, lipopeptides, glycopeptides, or analogs thereof. In the case of nonnatural peptides, one or more naturally-occurring amino acids can be substituted with a nonnatural amino acid such as, for example, a nonnatural amino acid containing isoprenyl or azide groups.

The peptides and methods described herein provide new strategies that can improve the oxidative folding of peptides while allowing accelerated analysis of the final folded product. In one aspect, the methods involve assigning the connectivity of two or more disulfide bridges in a peptide, wherein the method includes using NMR spectroscopy to (1) identify the position of a labeled residue responsible for disulfide formation in the peptide, and (2) identify the disulfide bridge that is associated with the labeled residue. In general, the methods use selectively labeled residues present in the peptide that are responsible for disulfide bridge formation and spectroscopically determining the position of the label(s) present in the specific disulfide bridge.

Disulfide bridges (—S—S—) are produced by the oxidative folding of two different thiol groups (—SH) present in the peptide. Thus, for example, if the peptide contains four different thiol groups (i.e., four amino acids each containing a thiol group), number of disulfide bridges can be formed within the peptide depending upon a variety of factors. The peptides prior to oxidative folding can be synthesized such that the exact position of the labeled residue can be ascertained. The term “labeled residue” is defined herein as a moiety that is the resulting product of the chemical species in a particular reaction scheme or subsequent formulation or chemical product, regardless of whether the moiety is actually obtained from the chemical species, wherein the residue can be identified spectroscopically. For example, a peptide that contains at least one —SH group can be represented by the formula Y—SH, where Y is the remainder (i.e., residue) of the amino acid. In this example, Y or a portion thereof is labeled.

In one aspect, the peptide can include cysteine, selenocysteine, norcysteine, or any combination thereof such that at least two disulfide bonds are formed upon oxidative coupling. It is contemplated that any combination of thiol containing compounds (e.g., amino acids) can be present in the peptide. For example, the peptide can include four cysteine residues, wherein at least one cysteine residue is labeled. In another aspect, the peptide has two labeled cysteine residues.

The methods described herein are not limited to ascertaining the exact position of multiple (i.e., greater than two) disulfide bonds present in the peptide. In one aspect, the number of thiol residues present in the peptide of interest is 2x, where x is an integer from 2 to 15, and the number of labeled thiol residues is x. In this aspect, there are from 2 to 15 disulfide bonds present, of which half of the thiol residues are labeled to some degree.

As described above, at least one of the thiol containing compounds (and residues in the peptide) is labeled so that it can be detected spectroscopically. In one aspect, NMR spectroscopy is used to evaluate the position of one or more labeled residues defined herein. In the case of amino acids, there are several options for labeling the amino acid. In one aspect, one or more carbon atoms of the amino acid can be ¹³C labeled. The amount of labeling can vary within the amino acid. For example, one specific carbon atom within the amino acid can be labeled with ¹³C (e.g., greater than 95%). In other aspects, all of the carbon atoms of the thiol-containing amino acid can be labeled with ¹³C (e.g., each carbon atom is greater than 95% ¹³C). In one aspect, the labeled amino acid is cysteine, wherein each carbon atom is labeled with about 95% or more ¹³C.

In other aspects, when the thiol-containing compound is an amino acid, one or more nitrogen atoms can be ¹⁵N labeled. For example, when the thiol-containing compound is cysteine, the nitrogen atom can be partially or completely labeled with ¹⁵N. In one aspect, when the thiol-containing compound is cysteine, each carbon atom of cysteine is labeled with about 20% or more ¹³C and the nitrogen atom of the labeled cysteine residue is ¹⁵N. In other aspects, when the thiol-containing compound is cysteine, each carbon atom is about 95% or more labeled with ¹³C and the nitrogen atom of the labeled cysteine residue is ¹⁵N.

It is also possible to use two different labels within the same peptide. For example, a first cysteine may have certain level or amount of labeling that is different from the amount of labeling in a second cysteine. In one aspect, at least one labeled cysteine residue has at least 95% or more of the carbon atoms labeled with ¹³C and the nitrogen atom of the labeled cysteine is ¹⁵N, and a second labeled cysteine residue has 20% or more of the carbon atoms labeled with ¹³C and the nitrogen atom of the second labeled cysteine is ¹⁵N.

In certain aspects, the peptide of interest includes at least two selenium residues that are capable of forming a diselenide bridge (—Se—Se—). Not wishing to be bound by theory, oxidative folding of peptides is improved with the formation of one or more diselenide bridges. As will be shown below in the Examples, peptides containing diselenide bridges are stable compounds that permit facile determination of the position of disulfide bridges present in the peptide. Additionally, the diselenide compounds are structural analogs to the corresponding disulfide compounds, which can be a source of potential drug candidates. The number of diselenide bridges present in the peptide can vary. In one aspect, the peptide has one or two diselenide bridges. In another aspect, the peptide has one or two diselenide bridges and from one to three disulfide bridges. In a further aspect, the peptide has one diselenide bridge and two disulfide bridges.

The diselenide bridge can be formed when two selenium compounds (e.g., compounds that contain a —SeH group) couple with one another. In one aspect, the diselenide bridge is derived from the coupling of any two of the following selenium-containing compounds present in the peptide: selenocysteine, homoselenocysteine, or norselenocysteine. In another aspect, the diselenide bridge is derived from the coupling of two selenocysteines present in the peptide. The incorporation of selenium compounds in the peptides can be performed using techniques known in the art. Exemplary methods for making these peptides are provided in the Examples.

In one aspect, any of the labeled peptides described herein can have at least one labeled disulfide bridge and at least one dicarba bridge. The term “dicarba bridge” is defined herein as a bridging group having the formula —CH₂CH═CHCH₂—. Examples of dicarba-containing cyclic peptides are described: J Med. Chem. 2009; 52(3):755-62. “Structure and activity of (2,8)-dicarba-(3,12)-cystino alpha-ImI, an alpha-conotoxin containing a nonreducible cystine analogue”; and in Chem Commun (Camb). 2009; (28):4293-5 “Regioselective formation of interlocked dicarba bridges in naturally occurring cyclic peptide toxins using olefin metathesis”) Similar to the diselenide bridge, the dicarba bridge can increase the stability of the peptide. Alternatively, the peptide can include at least one diselenide bridge and at least one dicarba bridge. In this aspect, the peptide can optionally contain one or more disulfide bridges, where one or more disulfide bridges may be labeled as described herein.

In other aspects, other optional groups can be present in the peptide to facilitate oxidative folding and subsequent characterization of the peptide. In one aspect, the peptide can further include at least two alkylfluoro groups. The term “fluoroalkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like, wherein at least one of the hydrogen atoms is substituted with a fluorine atom. A “lower fluoroalkyl” group is an alkyl group containing from one to six carbon atoms, wherein at least one of the hydrogen atoms is substituted with a fluorine atom. It is contemplated that some or all of the hydrogen atoms of the alkyl group can be replaced with fluorine. In one aspect, the fluoroalkyl group is a trifluoromethyl group.

The incorporation of the fluoroalkyl group in the peptide can be performed using techniques known in the art. For example, two cysteines normally used to produce a peptide can be substituted with a compound or amino acid that possesses a fluoroalkyl group. For example, the alkylfluoro group can be derived from 5,5,5,5′,5′,5′-hexafluoroleucine, perfluoro-norleucine, or perfluoro-norvaline.

The presence of two fluoroalkyl groups results in formation of a fluorous bridge within the peptide, which is a non-covalent interaction between the fluorine atoms of the two fluoroalkyl groups. The fluorous bridge can stabilize the peptide during and after oxidative folding. The fluorous bridge can also influence the specific formation of disulfide bridges. In the case when two thiol-containing compounds are replaced with fluoroalkyl compounds, fewer disulfide bridges are subsequently produced, which make the characterization of the peptide more straightforward. Finally, ¹⁹F NMR can be used to characterize the resultant peptide that contains the fluoroalkyl groups. It is contemplated that one or more fluorous bridges in combination with one or more disulfide and diselenide bridges can be present in the peptide.

In certain aspects, any of the peptides described herein can include one or more spacers. The spacers used herein are generally polymeric groups that can replace non-essential amino acids present in the peptide. In certain aspects, the spacer is inert and does not affect the overall activity of the peptide. In other aspects, the spacer can improve or enhance the biological activity of the peptide. This was demonstrated in Green et al. “Conotoxins Containing Nonnatural Backbone Spacers: Cladistic-Based Design, Chemical Synthesis, and Improved Analgesic Activity” Chemistry & Biology 14, 1-9, April 2007. In addition to enhancing activity of the peptide, the spacer can render the peptide more conformationally flexible and, thus, facilitate oxidative folding. Examples of spacers useful herein include, but are not limited to, a polyalkylene group, a polyether group, a polyamide group, a polyester group, a polyimino group, or a polythioether group. In another aspect, the spacer is 5-aminopentanoic acid, 5-amino-3-oxapentanoic acid, 6-aminohexanoic acid, 8-aminooctanoic acid, or 8-amino-3,6-dioxaoctanoic acid. The methods disclosed in Green et al. can be used to produce peptides having spacers and disulfide bridges.

In certain aspects, any of the peptides described herein can contain one or more N-substituted glycine residues, as present in peptoids (examples of peptoid are described in Simon R J, Kania R S, Zuckermann R N, Huebner V D, Jewell D A, Banville S, Ng S, Wang L, Rosenberg S, Marlowe C K, et al, Proc Natl Acad Sci USA. 1992; 89(20):9367-71 Peptoids: a modular approach to drug discovery; and Green, B. R., Bayudan, W., Ellison, M. E., Zhang, M. M., Yoshikami, D., Legowska, A., Rolka, K., Olivera, B. M., and Bulaj, G. (2006) Examples of conotoxin engineering: Introduction of non-peptidic backbone spacers into conotoxins and peptide-peptoid chimeras (Conopeptoids), Journal of Peptide Science 12, 209-209).

The labeled peptides described herein can be produced using standard techniques known in the art for producing peptides and related compounds. In the case of natural peptides, the peptide can be synthesized by recombinant methods, native chemical ligation methods, or a combination of chemical synthesis, semi-synthesis and recombinant methods. For example, by using recombinant methods or native chemical ligation bioorthogonal oxidation of the peptide may be beneficial for engineering growth factors, polypeptide-based hormones, antibody-derived therapeutics, such as miniantibodies, and other industrial proteins containing disulfide bridges. In one aspect, vectors containing cis-acting selenocysteine insertion sequences can be used to introduce simultaneously pairs of Sec-Sec and pairs of labeled Cys-Cys into recombinant polypeptides. In other aspects, SECIS, which permits the efficient recognition of the UGA stop codon, and engineered strains of host cells that can be used to produce the labeled peptides described herein.

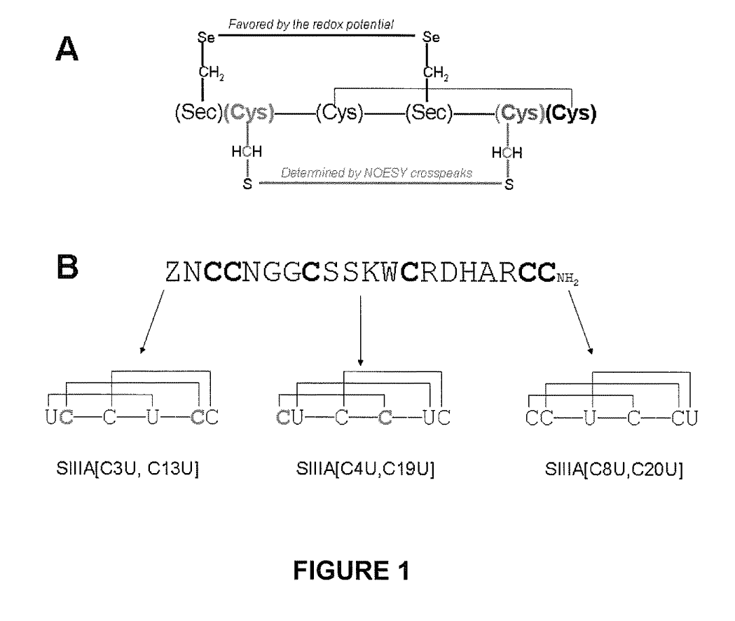

The position of the disulfide bridges produced by oxidative folding of the labeled peptides described herein can be unambiguously assigned using NMR spectroscopy. By knowing the exact position of the label in the peptide, it is possible assign which thiol-containing residue coupled with the labeled residue to form a disulfide bond. Depending upon the label used, numerous NMR techniques can be used to identify the labeled residue and ultimately the position of the disulfide bridges. In one aspect, when the labeled cysteine is ¹³C-labeled cysteine, the position of the labeled cysteine in the peptide can be identified by [¹³C,¹H] HSQC spectroscopy. In this aspect, once the position of the labeled cysteine has been identified, 2D ¹³C NOESY experiments can be conducted to unambiguously assign which disulfide bridge was formed by the labeled cysteine. Thus, by selectively labeling certain thiol-containing residues, the position of the disulfide bridges in the peptide can be assigned. An example of this is approach depicted in FIG. 1A, where the simultaneous use of selenocysteines and pairs of the ¹⁵N/¹³C labeled cysteine residues in μ-conotoxins and NMR spectroscopy can be used to identify the position of the two disulfide bridges in the peptide. Details regarding the assignment of the disulfide bridges in the peptide depicted in FIG. 1A are provided in the Examples.

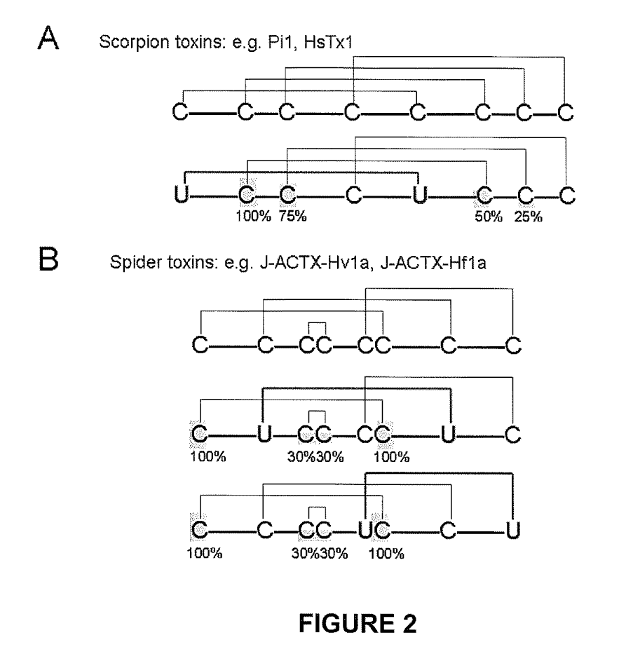

Since the oxidative folding of peptides rich with thiol groups (e.g., cysteine) is compatible with both chemical synthesis and recombinant expression methods, the labeled peptides and methods described herein have numerous applications, including the synthesis and analysis of combinatorial libraries, high-throughput parallel synthesis, characterization of novel cysteine-rich peptides, structural analysis of cysteine-rich peptides, lead optimization (structure-function-relationship analysis and improving PK/PD, bioavailability). FIG. 2 shows examples of how oxidative folding can be applied to the chemical synthesis of scorpion or spider toxins containing four disulfide bridges. A combination of diselenide bridges and differential labeling of individual cysteine residues may lead to peptides for which unambiguous assignment of the disulfide bridge connectivities could be achieved. Since the diselenide bridges are significantly more stable in the redox buffers, the disulfide/diselenide analogs may also appear very useful to study the role of individual disulfide bridges in the mechanism of oxidative folding (e.g., by measuring kinetics and thermodynamics of forming individual disulfide bonds in the context of pre-existing diselenide bridges). Thus, the methods described herein for evaluating oxidative folding in peptides should have significant effect on advancing research on disulfide-rich peptides and ultimately drug discovery and design.

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### I. Synthesis and Structural Characterization of μ-Selenoconotoxin Analogs of SIIIA

Chemical synthesis and folding. Peptides were synthesized using standard Fmoc [N-(9-fluorenyl)methoxycarbonyl] chemistry. The cysteine residues were protected with S-trityl groups, whereas the selenocysteine residues were protected with Se-4-metoxybenzyl groups. To remove all protected groups and cleave peptides from the resin in one step, the cocktail of TFA/thioanisole/phenol/water; (90:2.5:7.5:5, v/v/v/v)+1.3 eqv. DTNP [2,2′-dithiobis(5-nitropyridine)] was used (Hondal R., 2006). [Harris K M, Flemer S Jr, Hondal R J J Pept Sci. 2007 February; 13(2):81-93 Studies on deprotection of cysteine and selenocysteine side-chain protecting groups. After 3-4 h treatment with the cocktail, the peptides were filtered, precipitated and washed with cold methyl tert-butyl ether (MTBE). The peptides were dissolved in 0.01% (v/v) TFA in water and added to the mixture containing 50 mM DTT, 0.1 M Tris, 1 mM EDTA, pH 7.5. The reactions were carried out at room temperature for 1-2 h and then quenched with formic acid (8% final concentration). Afterwards the peptides were purified by reversed-phase HPLC using a semipreparative C18 Vydac column using a linear gradient from 5 to 30% buffer B in 25 min, where HPLC solutions were: buffer A-0.1% (v/v) TFA in water, and buffer B-0.1% (v/v) TFA in 90% aqueous acetonitrile (ACN). Oxidative folding of 1SeSe form of peptides were carried out in the presence of 0.1 M Tris-HCl, 1 mM EDTA (pH 7.5) and either 1 mM GSSG, 1 mM GSH or 1 mM GSSH, 10 mM GSH at 37° C. or room temperature. In addition, the folding of SmIIIA and SmIIIA[C3U,C15U] was performed in the presence of 1 M NaCl. To confirm if the diselenide bridge was preformed during the cleavage from the resin, the alkylation was carried out. For each 350 μl of conotoxin: SIIIA[C3U, C13U], SIIIA[C4U,C19U] and SIIIA[C8U,C20U] solution, 2 μl 4-vinylpyridine was added. The peptides were incubated in a dark place for 30 min at room temperature, then diluted with 150 μl 0.01% TFA in water, purified by HPLC and analyzed by mass spectroscopy.

Electrophysiological assays. Electrophysiological assays were carried out as described in Zhang M M et al, J Biol. Chem. 2007; 282(42):30699-706. Xenopus oocytes expressing Naᵥ1.2 were prepared and two-electrode voltage clamped with a holding potential of −80 mV. The membrane potential was stepped to a value between −20 and 0 mV (depending on Naᵥ subtype) for 50 ins every 20 sec. 3 μL of the peptide solution was applied to the 30 μL bath, and the bath manually stirred for about 5 seconds by gently aspirating and expelling ˜5 μL of the bath fluid several times with a micropipette. Peptide-exposures were in static baths to conserve material. Off-rate constants were determined from single-exponential fits of the time course of recovery from block during peptide-washout. All recordings were done at room temperature (˜21° C.).

NMR studies: Native peptides were prepared in a NMR buffer containing 40 mM sodium phosphate (pH 6.2), 50 mM sodium chloride, 90% H₂O and 10% D₂O. Resonance assignments were made using standard triple resonance NMR experiments, recorded as 2D versions, at 15° C. on a Varian Inova 600 MHz NMR spectrometer equipped with a cryogenic triple-resonance ¹H/¹³C/¹⁵N probe and z-axis pulsed-field gradients. Data were processed with FELIX 2004 (Accelrys, San Diego), and resonances assigned using standard approaches within the SPARKY program (T. D. Goddard and D. G. Kneller, University of California, San Francisco).

Labeling SIIIA[C3U,C13U]DOTA with terbium. The peptide (220 nmol) was dissolved in 220 μl nanopure water, then 40 μl of ammonium acetate buffer (pH 6.1) and 22 μl (0.01 M) of terbium (III) chloride hexahydrate were added. The mixture was shaken at 50° C. overnight. The progress of the reaction was checked with Xylenol Orange color test. After the reaction was completed, pH of the mixture was adjusted to 9 with NaOH (pH 12) and the solution was shaken for 30 min at 0° C. The peptide was centrifuged at 7000/min for 5 min, filtered with 0.22 μm filter, washed with nanopure water and concentrated in vacuum. The final product was analyzed by mass spectroscopy.

Synthesis of μ-selenocysteine analog. To obtain the very first proof-of-concept results, μ-SIIIA was selected, which is a three disulfide bridged conotoxin that exhibited relatively high yields during the direct oxidative folding (Bulaj G et al, Biochemistry. 2005; 44(19):7259-65). As illustrated in FIG. 1B, three SIIIA analogs were designed in which one pair of the cysteine residues forming the native disulfide bridge was replaced with the selenocysteine residues. The analogs were synthesized using the Fmoc-based chemistry on the automated peptide synthesizer. The cysteine thiols were protected with trityl groups, whereas the selenocysteine residues were protected with 4-metoxybenzyl groups. The protected groups were removed during the cleavage of the peptides from the resin. The Mob group came off easily with 1.3 eq DTNP and then the diselenide bridge was formed. The mechanism of the removal Mob group and closing the diselenide bridge was proposed by Hondal (J. Pept. Sci., 2007 Harris K M, Flemer S Jr, Hondal R J J Pept Sci. 2007 February; 13(2):81-93 Studies on deprotection of cysteine and selenocysteine side-chain protecting groups). The removal of the Mob group from selenocysteine residue showed how significant using the reducing reagent was, for example dithiothreitol, to remove the selenium-protecting 5-Npys group. Applying DTT to this reaction did not reduce the diselenide bridge because of the significantly higher redox potential (Eₒ=−323 mV) than the diselenide peptide (Eₒ=−381 mV). To confirm the existence of the preformed diselenide bridge in otherwise the reduced peptide, the alkylation with 4-vinylpyridine was carried out, followed by the HPLC separations and mass spectrometry analysis. The determined molecular masses of the alkylated products agreed with the calculated mass corresponding to the four alkylated cysteine residues and the closed diselenide bridge in SIIIA ([MH⁺]=2726.7): SIIIA[C3U,C13U]=2726.9, SIIIA[C4U,C19U]=2726.7, SIIIA[C8U,C20U]=2727.0. Thus, the apparent advantage of the bioorthogonal oxidation approach described here is an introduction of a pre-existing crosslink prior to the initiation of the folding; such crosslink can reduce the entropy of the unfolded peptide and may provide a favorable topography to form the remaining disulfide bridges.

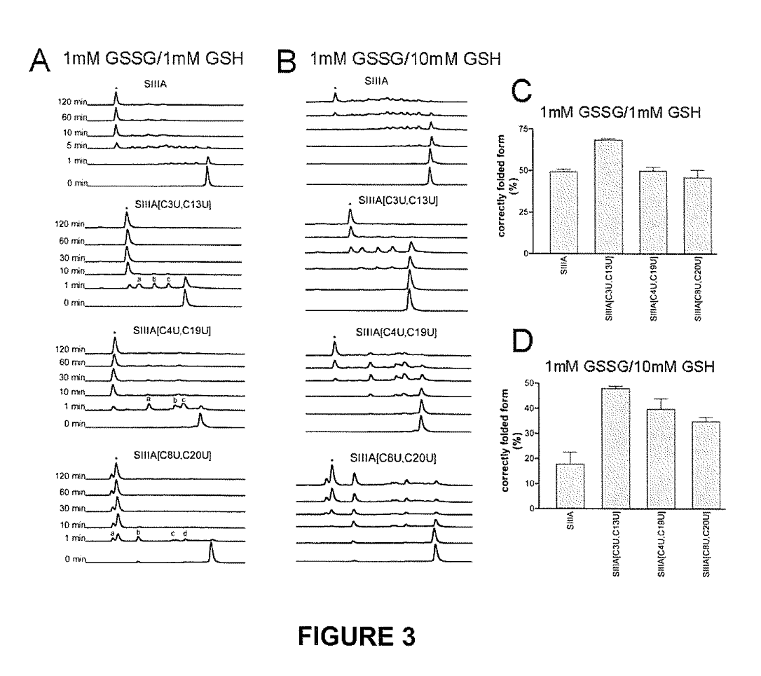

Next, the diselenide-containing peptides were subjected to the oxidative folding in the mixture of the reduced and oxidized glutathione that was previously shown to be optimized for the folding of mu-conotoxins (Fuller E et al, FEBS J. 2005; 272(7):1727-38). FIG. 3A shows the HPLC separations of the folding reactions carried out in the presence of 1 mM GSSG and 1 mM GSH, quenched after different reaction times. The yields for folding of mu-SIIIA and three analogs, SIIIA[C3U,C13], SIIIA[C4U,C19U] and SIIIA[C8U,C20U] were comparable, although the SIIIA[C3U,C13U] exhibited significantly higher yield, as compared to the wild-type and other two diselenide-containing analogs (FIG. 3C). The identity of each folded analog ([MH⁺]_{calc}=2302.7) was confirmed by MALDI-TOF: SIIIA[C3U,C13U] [MH⁺]ₑₓₚ=2302.5, SIIIA[C4U,C19U] [MH⁺]ₑₓₚ=2302.6, SIIIA[C8U,C20U] [MH⁺]ₑₓₚ=2302.4. Noteworthy, a number of the folding intermediates were much lower for all three diselenide-containing analogs, as compared to mu-SIIIA; none of the accumulated folding species contained a mixed disulfide with glutathione, as determined by mass spectrometry analysis. Using the folding redox conditions, 1 mM GSSG and 10 mM GSH, that are less favorable for the accumulation of the native species, the diselenide-containing analogs were tested to determine if they exhibit better thermodynamic stability, as compared to the all-disulfide containing mu-SIIIA. As shown in FIG. 3B and summarized in FIG. 3D, the highest steady-state accumulation of the native form was found for SIIIA[C3U,C13U] (48%), then slightly lower for SIIIA[C4U,C19U] and SIIIA[C8U,C20U], whereas mu-SIIIA exhibited the lowest thermodynamic stability. These findings are consistent with the previous data reported on a-selenoconotoxin ImI analogs that exhibited better thermodynamic stability, as compared to the native ImI that contained two disulfide bridges (Armishaw C. J. et al, J Biol. Chem. 2006; 281(20):14136-43). Thus, both double and triple crosslinked peptides showed superior stability when a diselenide bridge replaces one of the native disulfide bridges, suggesting that introduction of diselenide bridges may appear beneficial to a larger group of disulfide-rich peptides.

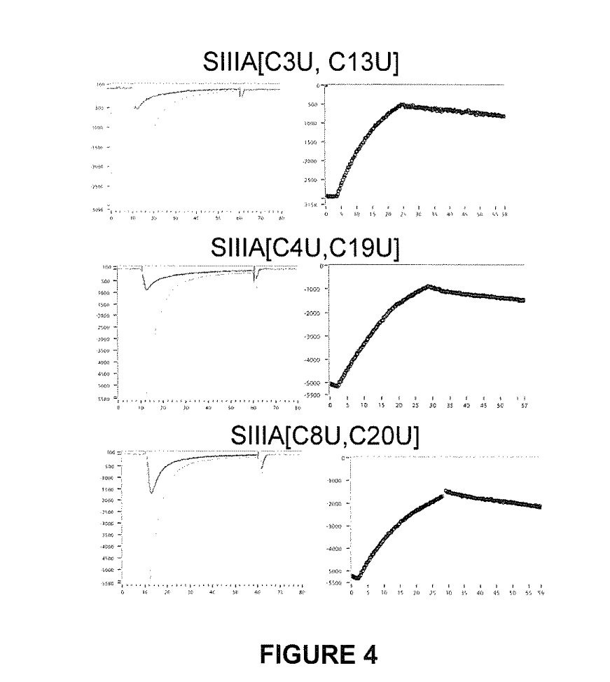

To investigate functional consequences of the disulfide to diselenide replacement, the SIIIA analogs were assayed for their activity to block Nav1.2 subtype of mammalian sodium channel. Since mu-SIIIA almost irreversibly inhibits Nav1.2, any structural changes from the apparent isomorphic replacement of the disulfide bridge would likely be reflected in the bioactivity of the analogs. As shown in FIG. 4, all three analogs exhibited comparable activity of blocking Nav 1.2, as compared to the wild-type conotoxin, although some subtle differences could be observed. 1 μM Solution of mu-SIIIA blocked Nav1.2 in 89±1.5%, whereas SIIIA[C3U,C13U], SIIIA[C4U,C19U] and SIIIA[C8U,C20U] inhibited the sodium currents in 84±1.9, 85±0.9 and 80±2.6%, respectively. These subtle differences between individual analogs might be in part accounted for by changes in bond distance (from S—S to Se—Se).

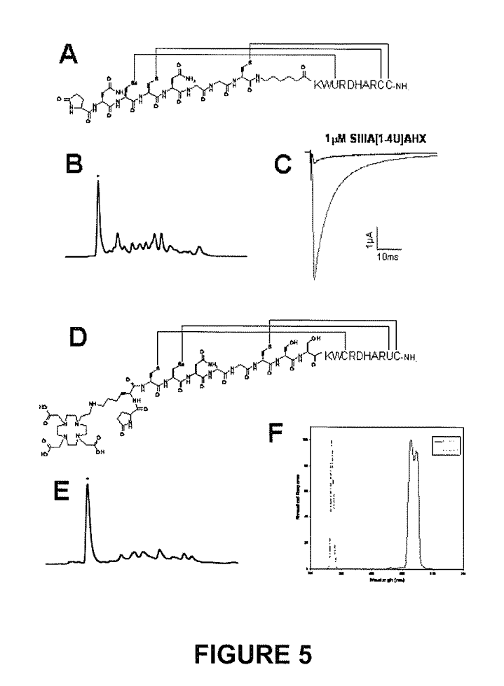

In attempt to test μ-selenoconotoxins in peptide engineering applications, two “nonnatural” selenoconotoxin SIIIA analogs were designed and synthesized: in one analog, two Ser residues in the second intercysteine loop were replaced by 6-aminohexanoic acid (backbone prosthesis), whereas the second analog contained Lys-DOTA at the N-terminus that would allow introduction of lanthanides into the peptide. Structures of both analogs are shown in FIG. 5. The oxidative folding of both analogs resulted in an accumulation of a major species (FIGS. 5B and 5E); both peaks were purified and subjected to further characterization. The backbone spacer-containing analog retained the ability to block Nav1.2 sodium currents (FIG. 5C), despite the fact that the backbone spacer was placed in the 2^{nd} intercysteine loop, adjacent to the critical residues. Similarly, the DOTA-SIIIA was active in the same assay. The DOTA-SIIIA was loaded with terbium: the excitation and emission spectra are shown in FIG. 5F, and the Tb-DOTA-SIIIA was as active as the analog without the lanthanide atom chelated. These results illustrate that the selenoconotoxins are useful in peptide engineering.

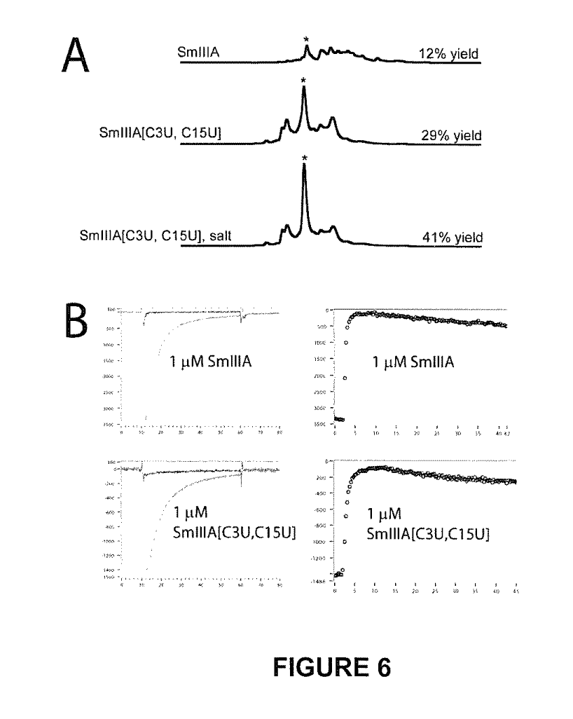

To test whether other μ-selenoconotoxins also exhibit improved folding properties, a pair of selenocysteines were introduced into μ-conotoxin SmIIIA:
ZRCCNGRRGCSSRWCRDHSRCC
   In contrast to μ-SIIIA, μ-SmIIIA was previously shown to fold with very low folding yields that ranged from 7% to 15%, depending on the folding conditions (Fuller E et al, FEBS J. 2005; 272(7):1727-38). As illustrated in FIG. 6A, the folding yield of the SmIIIA[C3U,C15U] was approximately 2.5-fold higher, as compared to all-Cys containing wild-type. Furthermore, when the folding reaction was carried out in the presence of 1 M NaCl and at low temperature (these folding conditions were used to optimized the folding of SmIIIA), the folding yield of the diselenide-containing SmIIIA further increased to 41%. Both, mu-SmIIIA and the selenoconotoxin analog blocked Nav1.2 sodium channels (FIG. 6B), confirming that the disulfide to diselenide replacement did not affect the bioactivity of mu-selenoconotoxins.

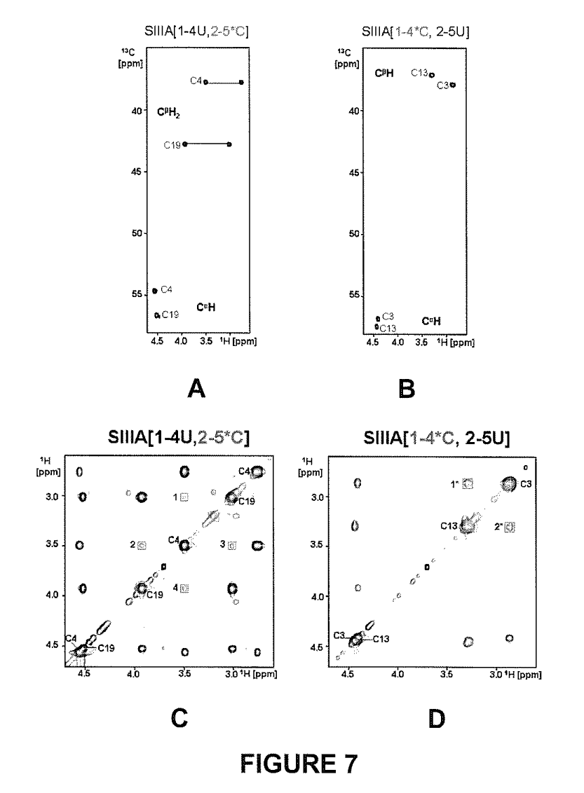

NMR-based analysis of the disulfide bridging. The position-specific introduction of the 15N/13C labeled Cys residues in μ-selenoconotoxin SIIIA analogs, (FIG. 1B), permits rapid acquisition of information as to whether a disulfide bridge was formed between two labeled Cys residues. The ¹³C/¹⁵N-labeled cysteines in the two μ-selenoconotoxin analogs, SIIIA[C3U,C13U,*C4,19] and SIIIA[*C3,13,C4U,C19U] analogs were identified using 2D [¹³C,¹H] HSQC experiments. The methine and methylene resonances were assigned in both analogs using the reported chemical shifts for μ-SIIIA (FIGS. 7A and 7B). Following resonance assignment, 2D ¹³C—F2-edited NOESY was recorded to identify cross-disulfide NOEs consistent with a disulfide bond. NOEs confirm the C4-C19 cystine, NOEs# 1-4 (rectangles), and C3-C13, NOEs# A and B (rectangles) (FIGS. 7C and 7D, respectively). Thus, it was possible to assign the proper connectivity of the crosslinks in the μ-selenoconotoxin analogs by: (1) performing the diselenide bridge, and (2) detecting cross-disulfide NOEs for one disulfide bond.

### II. Synthesis and Structural Characterization of ω-Selenoconotoxin Analogs of GVIA

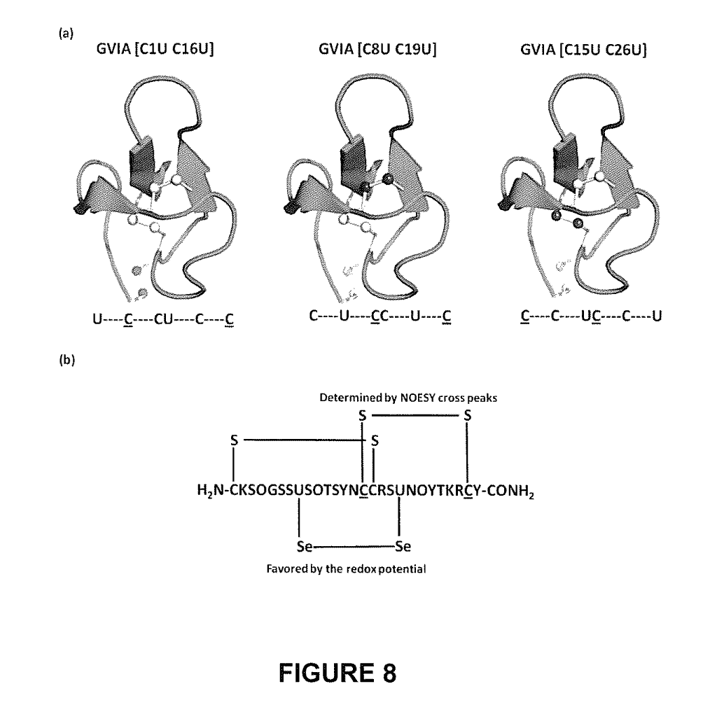

The oxidative folding of ω-conotoxin GVIA by site-specific incorporation of selenocysteine was investigated. To access the effect of selenocysteine in native form on oxidative folding, three analogs were synthesized, namely GVIA[C1U C16U], GVIA[C8U C19U] and GVIA[C15U C26U]. FIG. 8 shows Sec-GVIA analogs and strategy for disulfide connectivity determination. An integrated approach of disulfide mapping and biological assays were employed to determine the connectivity in folded peptide.

Peptide synthesis. Peptides were synthesized using standard Fmoc (N-(9-fluorenyl)methoxycarbony) chemistry and activated Opfp (pentafluorophenyl) esters of the protected amino acids. Side chains of selenocysteines were protected with para-methoxy benzyl group (ChemImpex International, Wood Dale, Ill.), labeled Cysteines (U-13C3, 97-99%; 15N, 97-99%; Cambridge Isotope Laboratories, Andover, Mass.) and unlabeled cysteines were protected with Trt group and hydroxy-proline was protected with tBu group. Sec-GVIA analogs were cleaved off from the resin using enriched reagent K {(trifluoroacetic acid (TFA)/thianisol/phenol/water (90:2.5:7.5:5) and 1.3 equivalents DTNP(2,2′-dithiobis(5-nitropyridine)} and GVIA was cleaved using reagent K {(TFA)/thianisol/phenol/water/ethanedithiol (82.5:5:5:5:2.5)}. Peptides were precipitated with methyl-tert-butyl ether (MTBE) and washed several times with cold MTBE. Sec-GVIA analogs were subjected for DTT (threo-1,4-dimercapto-2,3-butanediol) treatment, before purification, using 50 mM DTT in 100 mM Tris-HCl (pH 7.5) containing 1 mM EDTA for 1 hr. Peptides were purified using preparative RP-HPLC using Cᵢₛ column over a linear gradient of 10-35% Buffer B (90% acetonitrile containing 0.1% TFA) for 40 min. Purified peptides were analyzed using mass spectrometry. Observed mass of Sec-GVIA analogs is 2 Da less than predicted mass, confirming the presence of diselenide in the peptide (Expected: 3145 Da and observed: 3143 Da).

Oxidative folding. Folding reactions were initiated by resuspending 5 mmols of linear peptides into a 200 ul of folding buffer containing 0.1 M Tris-HCl (pH 7.5), 1 mM EDTA, 1 mM oxidized glutathione and 2 mM reduced glutathione. Reaction was quenched, after appropriate time interval, by acidification with formic acid (10% final concentration). Samples were further analysed using reverse-phase C₁₈ analytical HPLC over a linear gradient of 10-40% Buffer B (90% acetonitrile containing 0.1% TFA) in 40 mM. Accumulation of natively folded peptide at a given time point was calculated by integrating the HPLC chromatogram. Two Sec-GVIA analogs have nearly same retention time for folded and linear peptides, in such cases, the isotopic patterns of observed and predicted masses were compared and quality of folded peptides were accessed. At all the given time intervals, the observed isotopic pattern was nearly identical to the predicted peptide, confirming the presence of folded peptide. The most intense-peak of Linear and folded peptides in mass spectrum have same charge stated: [M+4H]⁴⁺. Experimental points were analyzed by prism software (GraphPad Software, Inc, San Diego, Calif.) and the rate constant was calculated by single exponential fit.

Mass spectrometry and NMR spectroscopy. Electrospray ionization (ESI) mass spectra were obtained using a Micromass Quattro II mass spectrometer. ESI-FTMS was recorded using Thermo-FT-MS and data were analyzed using the software provided by the manufacturer. Theoretical isotopic pattern of Sec-GVIA was using molecular formula-C₁₁₄H₁₉₀N₃₆O₄₃S₄Se₂Z₆X₂, where Z=¹³C and X=¹⁵N. Sec-GVIA analogs for NMR was prepared by dissolving purified peptide (1 mM) in buffer containing 40 mM sodium phosphate (pH 6.2), 50 mM sodium chloride, 90% H₂O, and 10% D₂O. Two-dimensional [¹³C, ¹H] HSQC and 2D [¹H, ¹H] NOESY were recorded at 15° C. on a Varian Inova 600 NMR spectrometer with a cryogenic ¹H/¹³C/¹⁵N probe. Data were processed with FELIX2004 and analyzed using SPARKY program.

Circular Dichroism Spectroscopy. Far-UV CD spectra were recorded with an AVIV Model 62D spectropolarimeter, using a bandwidth of 1 nm, a step size of 1 nM, and an average time of 0.5 sec. Linear peptides GVIA and Sec analogues were dissolved in phosphate buffer of pH 8.0 containing 1 mM DTT and samples were preincubated with buffer for 5 min before recording the spectra. Folded peptides GVIA and Sec-GVIA analogs were dissolved in phosphate buffer of pH 6.8 and subsequently recorded the spectra. All measurements were taken at room temperature, over 300-190 nm wavelength range using cell of 0.1 cm path length. Peptide concentration was 100 μM as determined by the absorbance at 280 nm and an extinction coefficient calculated from amino acid composition. Five independent spectra were collected for each sample and averaged. The contribution of buffer to the CD signal was eliminated by subtracting the peptide CD signal with that of the buffer CD signal. All Spectral intensities were expressed as mean residue elipticities.

Behavioral assay. Intracerebral injection of folded GVIA and Sec-GVIA analogs to 21-23 days old Swiss Webster mice were achieved using syringe with 29-gauge needle. Mice injected with equal volumes of normal saline were used as control. After injection, mice were placed in cage for observation. All the peptides exhibited shaking syndrome, which is characterized by persistent body trimmer and this behavior prolongs for long time in does dependent manner.

Chemical Synthesis and Oxidative folding. Site-specific incorporation of selenocystine was achieved by selectively introducing p-methoxybenzyl protected selenocysteine during solid-phase peptide synthesis. Selenocysteine at position Cys1/Cys16 (or) Cys8/Cys19 (or) Cys15/Cys26 of GVIA was introduced to yield three distinct native like connectivity in Sec-GVIA analogs. The crude peptide, obtained after cleavage from peptide resin, was subjected to 50 mM DTT treatment to enrich reduced conotoxin. Predicted reduced peptide mass of the Sec-GVIA analogs is 3145 Da and observed mass for GVIA[C1U C16U], GVIA [C8U C19U] and GVIA[C15U C26U] is 3143.2 Da, 3143.5 Da and 3143.2 Da, respectively. The observed mass for all the reduced Sec-GVIA analogs is 2 Da less than the predicted mass, indicating the presence of preformed diselenide bridge in these analogs. Mechanistic features underlying in deprotection of selenocysteine side chain protecting group by DTNP and the low redox-potential of diselenide (Eₒ=381 mV) over disulfide (Eₒ=180 mV)/selenyl sulfide (Eₒ=−326 mV) with respect to DTT (Eₒ=−323 mV) confirms the presence of preformed diselenide in the reduced Sec-GVIA analogs. The diselenide containing reduced Sec-GVIA analogs were subjected to oxidative folding in the mixture of oxidized (1 mM GSSG) and reduced (2 mM GSH) glutathione.

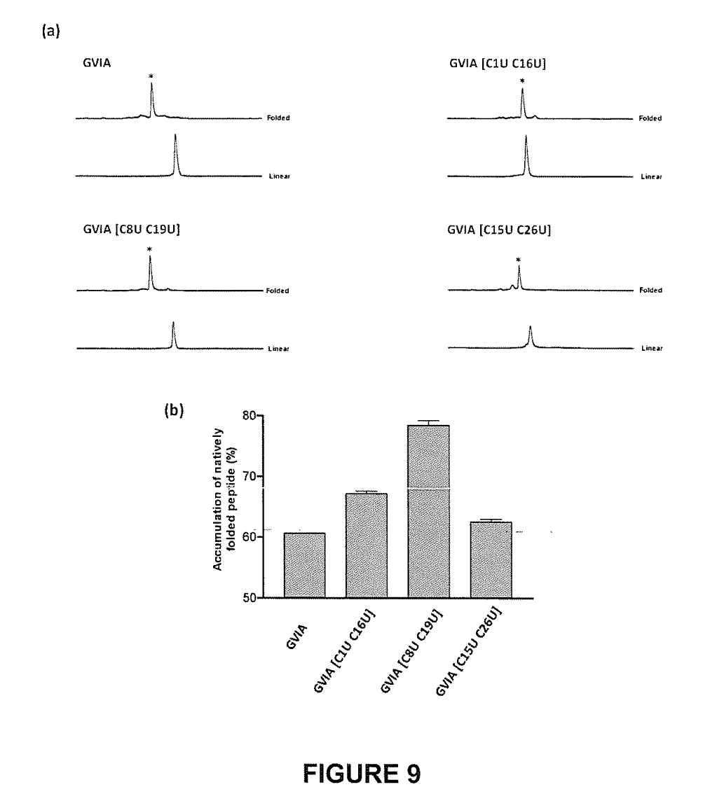

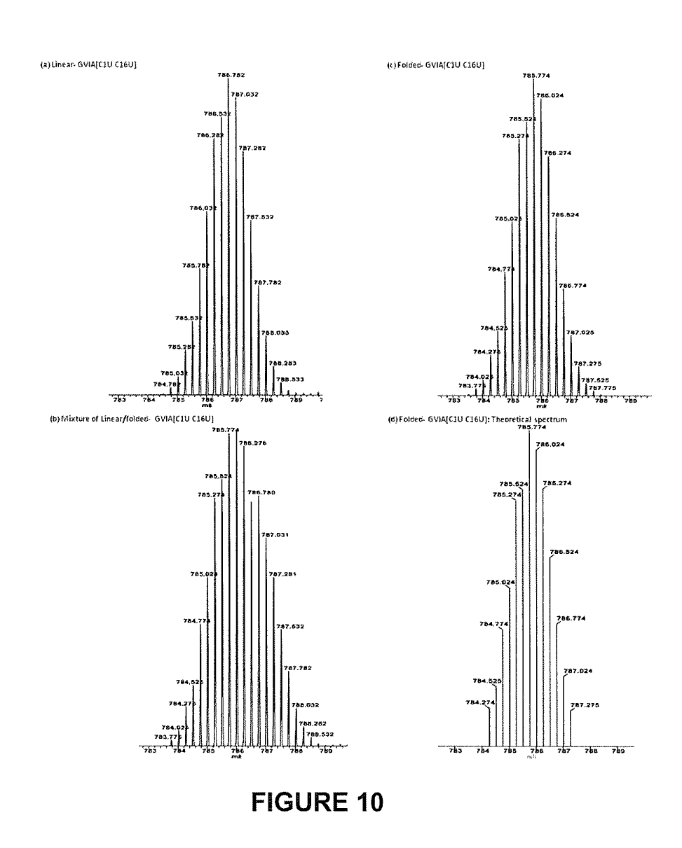

FIG. 9a shows chromatographic elution profiles of oxidative folding of GVIA and Sec GVIA analogues at steady-state. The identity of folded peptides was further characterized using mass spectrometry. Folded and reduced forms of GVIA[C1U C16U] have identical retention time pointing to the danger of presence of residual linear peptide in folded peptide fraction, which would be difficult to assess using regular average mass spectrum. In order to assess the quality of folded peptide GVIA[C1U C16U], high resolution FT-MS was employed and an isotopic pattern was generated from the elemental composition and compared to that of the observed peptide isotopic pattern. FIG. 10 shows high-resolution FT-mass spectrum of folded GVIA [C1U C16U] and the corresponding theoretical spectrum. The isotopic pattern of theoretical and experimental mass spectrum are nearly identical, confirming the absence of residual linear peptide in folded GVIA[C1U C16U]. The presence of residual reduced peptide would have altered the isotopic pattern in mass spectrum as indicated in the FIG. 10b, suggesting the use of high resolution FT-MS as a diagnostic tool in accessing the quality folded peptides having same retention time with that of reduced linear disulfide rich peptides. FIG. 9b shows the steady-state accumulation of natively folded GVIA and Sec-GVIA analogs during oxidative folding. The order of highest accumulation of natively folded peptides for Sec-GVIA analogs is GVIA [C8U C19U]>GVIA (C1U C16U]>GVIA [C15U C26U]. The folding efficiency of GVIA and GVIA [C15U C26U] was found to be the nearly same. Diselenide in second position of canonical disulfide connectivity of ω-conotoxin-GVIA has greater influence in improving the folding efficiency to yield native form.

Disulfide mapping using NMR spectroscopy. Disulfide mapping of Sec-GVIA analogs was achieved using an “integrated oxidative” folding approach, where the combination of diselenide and labeled cysteines were used in disulfide mapping. In the case of peptides containing three-disulfide bridges, the integrated approach of disulfide mapping mainly relies upon observation of cross-disulfide H^{α}/H^{β1}/H^{β2} NOESY cross-peak across selectively labeled cysteines. The preformed diselenide bridge restricts the possible number of disulfide connectivites in three disulfide containing peptides to three distinct possibilities. The site-specific incorporation of ¹⁵N/¹³C labeled Cys residues assists in rapid identification of cross-disulfide NOEs and hence to confirm the presence of the disulfide bridge between labeled Cys residues. Mass spectrometric data in conjunction with oxidative folding establish the disulfide bond between remaining Cys residues, confirming the overall folding pattern of the three disulfide containing peptides.

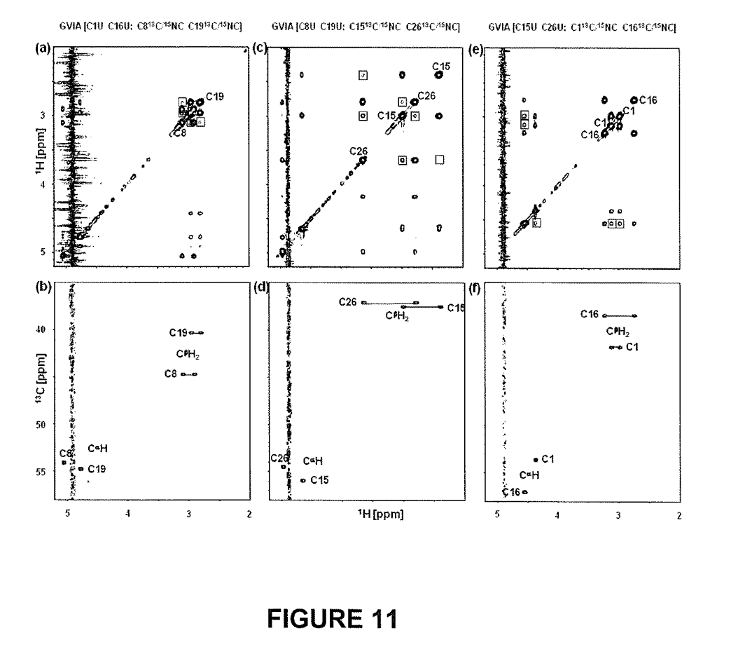

Sec-GVIA analogs contain a diselenide in native like connectivity at distinct positions in each analog. In order to achieve the disulfide mapping of these analogs, position-specific ¹⁵N/¹³C labeled cysteines were introduced during chemical synthesis. GVIA [C1U C16U] contains labeled cysteines at Cys-8 & Cys-19 position, GVIA[C8U C19U] contains labeled cysteines at Cys-15 & Cys-26 position, and GVIA[C15U C26U] contains labeled cysteines at Cys-1 & Cys-16 positions, respectively. FIG. 11 shows the hetero-nuclear NMR spectra of Sec-GVIA analogs. 2D [¹³C,¹H] HSQC experiments were performed to identify the labeled Cys residues. The corresponding methane and methylene protons were assigned using the reported chemical shift values of GVIA. 2D ¹³C—F2-edited NOESY were recorded to identify cross-disulfide NOEs across inter-residual H^{α}/H^{β1}/H^{β2} protons and confirm the disulfide bridge between labeled Cys residues. Two cross-peaks were observed across H^{β}/H^{β} protons of labeled cysteines in GVIA[C1U C16U], three cross-peaks were observed across H^{β}/H^{β} protons of labeled cysteines in GVIA[C8U C19U], and three cross-peaks were observed across H^{α}/H^{β} & H^{α}/H^{α} protons of labeled cysteines in GVIA[C15U C26U], which confirms the presence of a disulfide bond between labeled cysteine residues in Sec-GVIA analogs. In case of GVIA[C15U C26U], strong inter-residue H^{α}/H^{β} & H^{α}/H^{α} NOEs are present and H^{β}/H^{β} NOEs are absent, which may be due to the motional averaging about the disulfide (S—S) bond. The refined GVIA structure suggests that the disulfide Cys1-Cys16 exist in two different confirmations, indicating the motion about disulfide bond and consequently affecting the orientation of methylene protons. Mass spectrometric data of folded Sec-GVIA analogs confirms the formation of the remaining disulfide bridge during oxidative folding. Thus the disulfide connectivity in all the Sec-GVIA analogs is between Cys1-Cys16, Cys8-Cys19 and Cys15-Cys26, which is identical to that of disulfide pairing in GVIA. The oxidative folding of GVIA under the experimental conditions employed in folding is known to yield the native like disulfide connectivity.

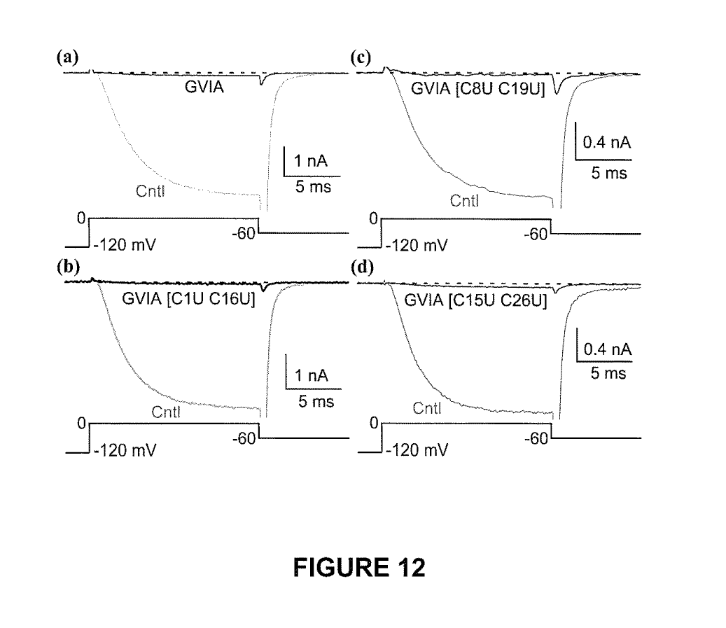

Electrophysiology and behavioral assay. FIG. 12 shows the blocking effectiveness of GVIA and Sec-GVIA analogs. N-type currents recorded were before (grey) and after (black) 16 min application of the conopeptides, which were GVIA (a), GVIA [C1U C16U] (b), GVIA [C8U C19U] (c), and GVIA [C15U C26U]. Voltage protocol is shown at the bottom of each panel. The tail currents on the control records were clipped to highlight the block of step current. Table 1 shows the comparison of the blocking effect among the ω-conotoxins. Data were calculated as % of blocked current following 16 min of 1 μM toxin application. Results are presented as mean±standard deviation.

TABLE 1

| Toxin | % Block | n |
|---|---|---|
| GVIA | 98.5 ± 1.0 | 4 |
| GVIA [C1U C16U] | 98.7 ± 0.1 | 4 |
| GVIA [C8U C19U[ | 96.6 ± 1.7 | 4 |
| GVIA [C15U C26U] | 96.5 ± 0.7 | 5 |

Intracranial injection of folded GVIA and Sec-GVIA analogs in mice exhibited shaking syndrome. Table 2 shows behavioral analysis of GVIA and Sec-GVIA analogs upon intracranial injection in mice. Injected mice were persistently shaking their body and this behavior continued a few minutes after the injection. Prolongation of persistence trimmer is observed to be dose dependent and at 1 nmol the behavior lasted for more than a day with mice being able to carry out normal functions along with shaking. At higher concentrations, mice also exhibited a passive behavior with the leg-extension in backwards. Similar behavioral features exhibited by GVIA and Sec-GVIA analogs upon intracranial injection in mice emphasize the isomorphic replacement of cysteine to selenocysteine.

TABLE 2
Effect of GVIA and Sec-GVIA analogs in exhibiting shaking
syndrome in mice upon intracranial injection* (*ability to induce
persistent shaking is not so prominent at <50 pimol).

| | Number | | | | |
|---|---|---|---|---|---|
| Does | of mice | | GVA [C1U | GVIA [C8U | GVIA [C15U |
| (pimol) | injected | GVIA | C16U] | C19U] | C26U] |
| 1000 | 1 | + | + | + | + |
| 500 | 1 | + | + | + | + |
| 250 | 1 | + | + | + | + |
| 100 | 1 | + | + | + | + |

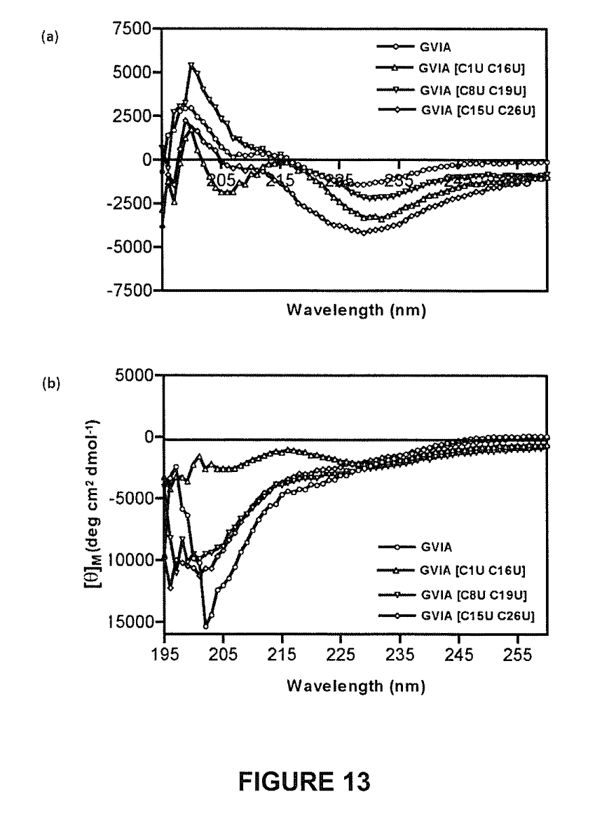

Characterization of GVIA and Sec-GVIA analogs using CD Spectroscopy. Specific bands in the far-UV circular dichroism spectra have been widely used for rapid determination of backbone conformation of proteins (or) peptides. Secondary structural information derived from CD spectra can represent qualitatively the overall fold of molecule and such studies have been extensively employed in the characterization of ω-conotoxin GVIA and its various analogs. FIG. 13a shows circular dichroism spectra of GVIA and Sec-GVIA analogs. The spectrum of GVIA is characterized by positive maximum at 200 nm and broad minimum around 230 nm, consistent with β-sheet structure as derived from NMR spectroscopy. Sec-GVIA analogs also displayed similar structural features with that of GVIA, indicating the overall conserved fold and successful folding to yield the native like disulfide/diselenide bridge between respective cysteine and selenocysteine residues.

In the case of GVIA[C1U C16U], there is a deep minima around 205 nm and a strong maxima around 200 nm. Such features also present in linear-GVIA[C1U C16U]. However, the disulfide mapping and activity studies strongly support the native like connectivity and overall fold of GVIA[C1U C16U]. Linear Sec-GVI analogs were subjected for CD analysis to investigate the presence of any secondary structural feature at a diselenide containing peptides. FIG. 13b shows far UV circular dichroism spectra of linear Sec-GVIA analogs in the presence of 1 mM DTT. For comparison, a spectrum of linear GVIA containing free thiols was also shown. CD spectra of linear GVIA[C8U C19U] and GVIA[C15U C26U] contain deep minima around 200 nm and no maxima, which resembles unstructured peptide. For the analog GVIA[C1U C16U], the CD spectrum suggests the presence of some residual regular structure or may be spectral artifact of the peptide as evident from the corresponding folded peptide. CD spectra of folded GVIA and Sec-GVIA analogs along with their corresponding linear peptides clearly indicates the direct contribution of disulfides in stabilizing the native like fold. A noteworthy observation is the analog linear-GVIA[C8U C19U], which has greater influence in improving the folding yield, is unstructured and resembles random-coil with that of linear-GVIA.

Folding kinetics of GVIA and Sec-GVIA analogs. Sec-GVIA analogs contain a diselenide bridge in distinct positions with native like connectivity resulting in variable spacing of remaining thiol groups with respect to diselenide loop. The restriction imposed by the diselenide bond on folding and its subsequent effect on the orientation of the thiol groups was assessed by oxidative folding using redox buffer containing mixture of oxidized/reduced glutathione. Oxidative folding of GVIA and Sec-GVIA analogues were initiated by adding linear peptides into redox buffer and the reaction was quenched at a particular time point by acidification using formic acid. The quenched reaction was analyzed using chromatography and separated peaks were further characterized by mass spectrometry. The accumulation of folded peptide was determined by integrating the chromatographic peaks in the background of other reaction intermediates and the quality of folded peptide was further assessed using isotopic pattern derived from high resolution mass spectra.

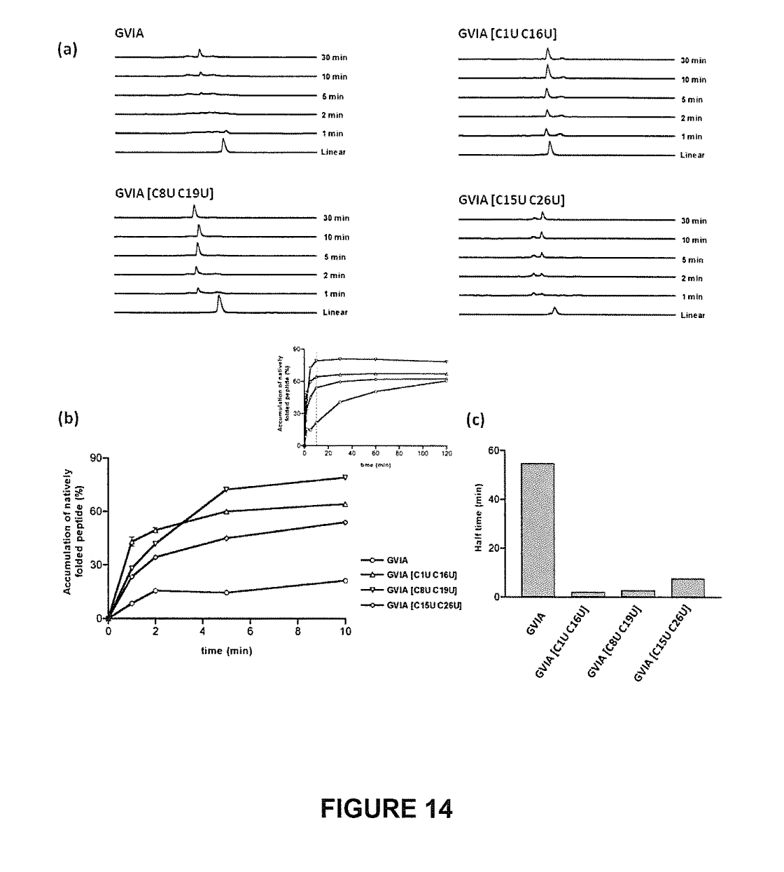

FIG. 14a shows RP-HPLC elution profile of the oxidative folding of GVIA and Sec-GVIA analogs at regular time intervals. Accumulation of natively folded peptide was quite rapid in Sec-GVIA analogs compared to that of GVIA, which indicates the close proximity of thiol groups in Sec-GVIA analogs. However, Sec-GVIA analogs required formation of the remaining two disulfide bonds and GVIA required the formation of three-disulfide bonds. The participation of diselenide in glutathione mediated exchange reaction in Sec-GVIA analogs also cannot be ruled out. Oxidative folding of GVIA[C15U C26U], at early folding intervals, resulted in significant accumulation of scrambled isomer, which later decreased in intensity with an increase in the amount of natively folded peptide. The observed mass for two majorly populated peaks in early folding time points of GVIA[C15U C26U] was 3139.8 Da and 3139.6 Da, respectively. This observation supports differential orientation of the thiol groups in Sec-GVIA analogs.

FIG. 14b shows the folding kinetics of the accumulation of natively folded peptide in GVIA and Sec-GVIA analogs. Accumulation of natively folded peptide in Sec-GVIA analogs reached equilibrium in one hour compared to GVIA under the conditions described in experimental procedure. Rate constant (kₒₙ) for accumulation of natively folded peptide in GVIA [C1U C16U] is 19.0 m⁻¹M⁻¹, in GVIA [C8U C19U] is 18.0 0 m⁻¹M⁻¹, and in GVIA [C15U C26U] is 0 m⁻¹M⁻¹. It is evident from FIG. 14b the rate of accumulation of folded peptide in GVIA [C1U C16U] was rapid compared to the rest of Sec-GVIA analog, which contains maximum number of residues (15 residues) within the diselenide loop. The analogs GVIA[C8U C19U] and GVIA[C15U C26U] have the identical number of residues within the diselenide loop, however, the former peptide is known to orient the thiol groups in close proximity to yield miss-folded peptide.

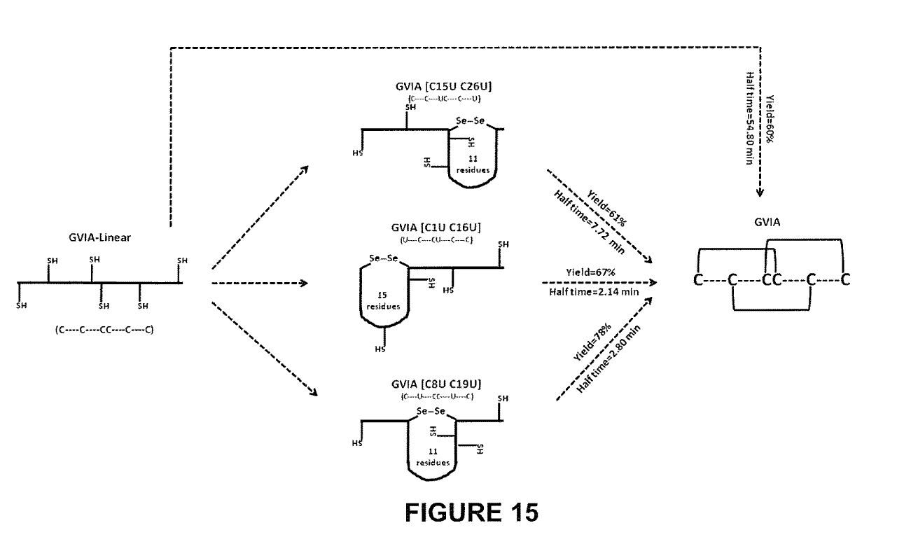

The effect of preformed diselenide on oxidative folding of GVIA was further emphasized in FIG. 14c. The kinetic experiments clearly demonstrate the restriction imposed by the diselenide on oxidative folding of ω-conotoxin GVIA and also emphasize the role of diselenide in decreasing the randomness of polypeptide chain. Degrees of limitations imposed by the diselenide bond on randomness of the peptide chain and the orientation of thiol groups are the cause for variable folding yields of Sec-GVIA analogs. FIG. 15 summarizes the results derived from the oxidative folding of GVIA and Sec-GVIA analogs. In conclusion, site-specific incorporation of selenocysteine in native like connectivity (1) decreases folding time to yield native like connectivity, (2) exhibits differential influence on folding efficiency, (3) affects the orientation of thiol groups and (4) not always associated in improving folding yield.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A peptide comprising at least two disulfide bridges, wherein at least one residue of one of the disulfide bridges is labeled.

2. The peptide of claim 1, wherein the peptide comprises at least one diselenide bridge.

3. The peptide of claim 1, wherein the peptide comprises one diselenide bridge.

4. The peptide of claim 1, wherein the peptide comprises a pair of diselenide bridges.

5. The peptide of claim 2, wherein the diselenide bridge is derived from the coupling of any two of the following selenium-containing compounds present in the peptide: selenocysteine, homoselenocysteine, or norselenocysteine.

6. The peptide of claim 2, wherein the diselenide bridge is derived from the coupling of two selenocysteines present in the peptide.

7. The peptide of claim 1, wherein the disulfide bridge is derived from the coupling of two thiol residues comprising cysteine, selenocysteine, norcysteine, or any combination thereof present in the peptide, wherein at least one of the thiol compounds is labeled.

8. The peptide of claim 1, wherein each disulfide bridge is derived from the coupling of two cysteine residues present in the peptide, wherein at least one cysteine residue comprises a labeled cysteine.

9. The peptide of claim 7, wherein the number of thiol residues comprises the formula 2×, where x is an integer from 2 to 15, and the number of labeled thiol residues is x.

10. The peptide of claim 8, wherein each carbon atom of the labeled cysteine residue is labeled with about 95% or more ¹³C.

11. The peptide of claim 8, wherein the nitrogen atom of the labeled cysteine residue is ¹⁵N.

12. The peptide of claim 8, wherein each carbon atom of the labeled cysteine residue is labeled with about 95% or more ¹³C and the nitrogen atom of the labeled cysteine residue is ¹⁵N.

13. The peptide of claim 8, wherein each carbon atom of the labeled cysteine residue is labeled with about 20% or more ¹³C and the nitrogen atom of the labeled cysteine residue is ¹⁵N.

14. The peptide of claim 8, wherein each carbon atom of at least one labeled cysteine residue is labeled with about 95% or more ¹³C and the nitrogen atom of the labeled cysteine is ¹⁵N, and each carbon atom of second labeled cysteine residue is labeled with about 20% or more ¹³C and the nitrogen atom of the second labeled cysteine is ¹⁵N.

15. A peptide comprising at least four cysteine residues, wherein at least one of the cysteine residues comprises a labeled cysteine.

16. The peptide of claim 15, wherein the peptide comprises at least two selenocysteines residues.

17. The peptide of claim 15, wherein the peptide comprises two labeled cysteine residues.

18. The peptide of claim 15, wherein the number of cysteine residues comprises the formula 2x, where x is an integer from 2 to 15, and the number of labeled cysteines is x.

19. A peptide comprising at least one labeled disulfide bridge and at least one dicarba bridge.

20. A peptide comprising at least one diselenide bridge and at least one dicarba bridge.

21. The peptide of claim 1, wherein the peptide further comprises at least two alkylfluoro bridges.

22. The peptide of claim 21, wherein the alkylfluoro group comprises a C₁-C₆ alkyl group, wherein at least one of the hydrogen atoms of the alkyl group is substituted with fluorine.

23. The peptide of claim 21, wherein the alkylfluoro group comprises a C₁-C₆ alkyl group, wherein all of the hydrogen atoms of the alkyl group are substituted with fluorine.

24. The peptide of claim 21, wherein the alkylfluoro group is a trifluoromethyl group.

25. The peptide of claim 21, wherein the alkylfluoro group is derived from 5,5,5,5′,5′,5′-hexafluoroleucine, perfluoro-norleucine, or perfluoro-norvaline.

26. The peptide of claim 1, wherein the peptide comprises at least one spacer.

27. The peptide of claim 21, wherein the spacer comprises a polyalkylene group, a polyether group, a polyamide group, a polyester group, a polyimino group, or a polythioether group.

28. The peptide of claim 1, wherein the peptide comprises a natural protein, a peptoid, a lipopeptide, or a glycopeptides, or analogs thereof.

29. The peptide of claim 1, wherein the peptide contains nonnatural amino acids containing isoprenyl or azide groups.

30. The peptide of claim 1, wherein the peptide is produced by recombinant methods, native chemical ligation methods, or a combination of chemical synthesis, semi-synthesis and recombinant methods.

31. The peptide of claim 1, wherein the peptide comprises one or more N-substituted glycine residues.

32. A method for assigning the connectivity of two or more disulfide bridges in a peptide of claim 1, the method comprising using NMR spectroscopy to (1) identify the position of the labeled residue in the peptide, and (2) identify the disulfide bridge that is associated with the labeled residue.

33. The method of claim 32, wherein when the labeled peptide comprises ¹³C-labeled cysteine, step (1) comprises identifying the position of the labeled cysteine in the peptide by [¹³C,¹H] HSQC spectroscopy.

34. The method of claim 33, wherein after identifying the position of the labeled cysteine, identifying which labeled cysteine is present in each disulfide bridge by 2D ¹³C NOESY experiments.
